(19) United States
(12) Patent Application Publication
(10) Pub. No.: US 2023/0398581 A1
(43) Pub. Date: Dec. 14, 2023

(54) Fluid Delivery System for a Vehicle

(71) Applicant: Nicolas Nocera, Port Richey, FL (US)

(72) Inventor: Nicolas Nocera, Port Richey, FL (US)

(21) Appl. No.: 17/836,054

(22) Filed: Jun. 9, 2022

(51) Int. Cl. B08B 3/02; B08B 1/00; B08B 13/00; B60R 15/00; A61L 2/26; A61L 2/00

(52) U.S. Cl. CPC B08B 3/02; B08B 1/002; B08B 13/00; B60R 15/00; A61L 2/26; A61L 2/0088; B67D 1/0003

(57) ABSTRACT

A fluid delivery system for a vehicle includes at least one reservoir, at least one ejector to facilitate an ejection of a fluid, at least one pump configured to pump at least one liquid from the at least one reservoir to the at least one ejector, and at least one valve arranged in fluid communication with the at least one pump and the least one ejector and adapted to control a flow of the at least one liquid from the at least one pump to the at least one ejector.

### FIELD OF INVENTION

The present disclosure relates generally to automotive vehicles. More particularly, the present disclosure pertains to a fluid delivery system mounted to a vehicle.

### BACKGROUND OF INVENTION

As a society, people spend a large amount of time driving, with current polls showing an average driver spends just under 300 hours per year behind the wheel, with solo trips constituting the vast majority of trips. (The WANADA Bulletin #35-16; 2016 Sep. 13). Further, most drivers will refill the fuel more than once per week. (Id.). As such, drivers and passengers frequently get into and out of vehicles and are regularly exposed to bacteria, dirt, and viruses. Moreover, drivers and passengers who enter the vehicle can contaminate the interior of the vehicle with the dirt and microbes on their hands and shoes. The interiors of vehicles can easily and unknowingly become prone to harbor and spread disease as well as becoming visibly unclean.

Systems have been designed to provide for a hand sanitizing system on the dash of the vehicle. (See, Gage, P. G. Pub. 2021/100406; Thibideau, U.S. Pat. No. 11,197,588). Similarly, disinfection systems have been mounted in or on the rearview mirror, with an automatic or manual dispenser. (Clair-Chalupka, U.S. Pat. No. 9,758,101; Baker, et al., U.S. Pat. No. 11,292,393). Other systems coat hand-contact surfaces in the vehicle in a disinfectant by seeping the disinfectant liquid through a nozzle onto the surface. (Paul, et al., DE 102017211515). However, these systems require the driver and passenger to enter the vehicle prior to accessing the system, allowing the interior of the vehicle to become contaminated. The system also fails to address the microbes, dirt and debris on the driver's or passenger's footwear.

Alternative systems have been designed to place a vehicle disinfection system in an exterior mirror, and using an RFID or NFC (near field communication) signal to prevent unauthorized use of the system. (Humpenoeder, et al., DE 102020004868). The system can also include a UV-A light, such as a 365 nm wavelength, to allow a user to visualize germs on the hands. (Id.).

Around 70% of Americans live in an environment that can result in snow and ice accumulating on the vehicle. A vehicle can become inaccessible due to the gathering of snow or ice on the vehicle. Exposure to extreme cold temperatures is undesirable, and therefore, reducing the time and effort to gain access to the vehicle is needed. Typical solutions involve heating the windows of the vehicle or vehicle parts, through heated air or a heating element. (Delorme, et al., DE 102011090327; Hua, et al., CN 114312258; Zubrecki, CA 2657052). However, during cold weather conditions, batteries and energy generation in a vehicle can be critical, due to loads placed on the vehicle electrical systems. Thus, each of these systems is suboptimal as each is energy-intensive.

The current systems fail to address the bacteria, viruses, dirt, and debris entering the vehicle, as well as dirt or snow and ice on the exterior of the vehicle. As such, a system is needed to ensure cleanliness and sanitary conditions in a vehicle.

### SUMMARY OF THE INVENTION

A fluid delivery system is disclosed herein, where the fluid delivery system includes at least one reservoir, at least one pump, at least one ejector, at least one valve arranged in fluid communication with the at least one pump and adapted to control a flow of the at least one fluid from the at least one pump to the at least one ejector, and a controller coupled to the at least one pump and the at least one valve. Specific embodiments of the invention include a sensor disposed on an exterior panel of the vehicle, and transmits an activation signal to the controller upon activation of the sensor thereby activating the fluid delivery system. The sensor is optionally a motion sensor, an ultrasonic sensor, or a passive infrared sensor. The fluid delivery system may include a manifold in fluid communication with the at least one reservoir, wherein the manifold comprises an input port and a plurality of output ports in fluid communication with the input port. The manifold is configured to split fluid between the plurality of output ports.

Fluid transport lines are optionally used to fluidly connect the constituents of the fluid delivery system. The fluid transport lines may be made of bendable plastic. Nonlimiting examples include acrylonitrile butadiene styrene (ABS), high impact polystyrene (HIPS), acrylic (PMMA), cellulose acetate, cyclic olefin copolymer (COC), ethylene-vinyl acetate (EVA), ethylene vinyl alcohol (EVOH), polyvinylfluoride (PVF), polyvinylidene fluoride (PVDF), polytetrafluoroethylene (PTFE), polychlorotrifluoroethylene (PCTFE), fluorinated ethylene-propylene (FEP), perfluoroalkoxy polymer (PFA), polyethylenechlorotrifluoroethylene (ECTFE), polyethylenetetrafluoroethylene (ETFE), perfluoropolyether (PCPE), acrylic/PVC polymer, aromatic polyester polymers (liquid crystal polymer), polyoxymethylene (acetal), polyamide (PA, nylon), polyamide-imide (PAI), polyaryletherketone (PAEK), polybutadiene (PBD), polybutylene (PB), polybutylene terephthalate (PBT), polycaprolactone (PCL), polychlorotrifluoroethylene (PCTFE), polyethylene terephthalate (PET), polycyclohexylene dimethylene terephthalate (PCT), polycarbonate (PC), polyhydroxyalkanoate (PHA), polyketone (PK), polyester, polyethylene (PE), polyetheretherketone (PEEK), polyetherimide (PEI), polyethersulfone (PES), chlorinated polyethylene (CPE), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), poly sulfone (PSU), polytrimethylene terephthalate (PTT), polyurethane (PU), polyvinyl acetate (PVA), polyvinyl chloride (PVC), polyvinylidene chloride (PVDC), and styrene-acrylonitrile (SAN).

The one or more reservoirs are configured to retain a fluid and has at least an output. However, certain configurations of the reservoir or reservoirs have an input to add fluid to the reservoir and an output, leading to the pump. Optional embodiments of the fluid delivery system include a first reservoir to store a first fluid and a second reservoir to store a second fluid. In these embodiments, a first pump configured to pump the first fluid from the first reservoir and the second reservoir is in fluid communication with the least one ejector and in fluid communication with a flow of the first fluid.

The pump or pumps of the fluid delivery system are in fluid communication with the output of the at least one reservoir and configured to pump the fluid contained in the reservoir. Where the fluid delivery system uses more than one reservoir, the system optionally includes a second pump in fluid communication with the second reservoir, and configured to pump the second fluid from the second reservoir. In embodiments using the second pump, the second pump is coupled to the controller to activate flow of the second fluid into the first fluid upon activation of the fluid delivery system by a user.

The ejector or ejectors of the fluid delivery system are in fluid communication the at least one pump, allowing for ejection of fluid from the fluid delivery system, and may be disposed at various locations in the vehicle based on the use, as provided.

The ejector or ejectors are optionally a hand wash ejector, foot wash ejector, harm deterrent ejector, defrost ejector, or a mix of ejectors. To facilitate the listed ejectors, the fluid delivery system includes a mixing valve or mixing chamber disposed between the first reservoir and the second reservoir, and in fluid communication with the first reservoir and the second reservoir to permit mixing of a first fluid and a second fluid. The embodiment of fluid delivery system also includes an output line in fluid communication with the at least one pump and an ejector disposed on the wash line and configured to eject a mixture of the first fluid and the second fluid liquid, where the second fluid is an alcohol or alcohol-based disinfecting fluid.

Options including the hand wash ejector include one or more ejectors, where an ejector is disposed on an exterior face of a vehicle body panel, an exterior vehicle mirror, a vehicle fueling port, a vehicle charging port, or a vehicle bed. The ejector includes a nozzle or port disposed on an end of the output line of the fluid delivery system and adapted to provide a spray of fluid. The system further includes a switch, button, or trigger disposed adjacent to the hand wash ejector and in electrical communication with the controller, allowing for activation and deactivation of the fluid flow.

In variations of the fluid delivery system having the foot wash ejector, the ejector is disposed on an exterior face of a vehicle door sill, an exterior vehicle wheel well, an exterior bumper, or a combination thereof. A nozzle or port disposed on an end of the output line of the fluid delivery system and adapted to provide a spray of fluid and includes a switch, button, or trigger disposed adjacent to the foot wash ejector and in electrical communication with the controller, allowing for activation and deactivation of the fluid flow.

In variations of the fluid delivery system having the harm deterrent ejector, the ejector is disposed on an exterior face of a vehicle body panel. A nozzle or port disposed on an end of the output line of the fluid delivery system and adapted to provide a spray of fluid, and includes a switch, button, or trigger in radio communication, electromagnetic communication, or electrical communication with the controller.

In variations of the fluid delivery system having the at least one defrost ejector, the ejector is disposed on an exterior face of a vehicle body panel and configured to eject a mixture of the first fluid and the second fluid liquid. A nozzle or port disposed on an end of the output line of the fluid delivery system and adapted to provide a spray of fluid. A switch, button, or trigger is provided and in radio communication, electromagnetic communication, or electrical communication with the controller, permitting activation and deactivation of the fluid flow.

The ejector or ejectors are alternatively at least one user wash ejector, fire retardant ejector, at least one vehicle wash system, at least one cloth washing ejector, at least one eye wash ejector, at least one drinking ejector, or at least one ice maker. In these embodiments, the fluid delivery system includes an output line in fluid communication with the at least one pump and an ejector disposed on the wash line and configured to eject the first fluid.

In variations of the fluid delivery system having the user wash ejector, the ejector is disposed on an exterior body panel of the vehicle or in a compartment disposed on the exterior body panel. A nozzle or port disposed on an end of the output line of the fluid delivery system and adapted to provide a spray of fluid, and a switch, button, or trigger disposed adjacent to the at least one cloth washing ejector and in electrical communication with the controller, allowing for activation and deactivation of the fluid flow.

Where the fluid delivery system includes the at least one fire retardant ejector, the ejector is disposed in a motor compartment or interior passenger compartment and aligned to provide a fluid to a predetermined location in the vehicle or around the vehicle. A nozzle or port disposed on an end of the output line of the fluid delivery system and adapted to provide a spray of fluid, and includes a switch or sensor in electrical communication with the controller. The system optionally includes a chemical fire retardant in fluid communication with the at least one fire retardant ejector.

Where the fluid delivery system includes at least one vehicle wash ejector, the ejectors are disposed on a plurality of exterior panels of the vehicle and aligned to provide a fluid substantially on the exterior of the vehicle. Optionally, the vehicle wash ejectors are rotatably disposed on the exterior panels of the vehicle, extendably disposed on the exterior panels of the vehicle, or a combination of the above options. A nozzle or port disposed on an end of the output line of the fluid delivery system and adapted to provide a spray of fluid, and includes a switch, button, or trigger in electrical communication with the controller. In specific variations of this embodiment, the fluid delivery system includes a mixing valve or mixing chamber disposed between the first reservoir and the second reservoir, and in fluid communication with the first reservoir and the second reservoir to permit mixing of a first fluid and a second fluid.

Where the fluid delivery system includes the at least one cloth washing ejector, the ejector is disposed on an exterior panel of the vehicle and aligned to provide fluid to a user. A nozzle or port disposed on an end of the output line of the fluid delivery system, and includes a switch, button, or trigger disposed adjacent to the at least one cloth washing and in electrical communication with the controller. Similarly, where the system includes the eye wash ejector, the ejector is disposed on an exterior body panel of the vehicle, in a compartment disposed on the exterior body panel, or an interior passenger compartment, and has a switch, button, or trigger disposed adjacent to the at least one eye wash ejector and in electrical communication with the controller to allow a user to trigger activation of the fluid delivery system. A nozzle or port disposed on an end of the output line of the fluid delivery system to facilitate delivery of the fluid. Likewise, where the fluid delivery system includes at least one drinking ejector is disposed on an interior passenger compartment, with a switch, button, or trigger disposed adjacent to the at least one drinking ejector and in electrical communication with the controller. A nozzle or port disposed on an end of the output line of the fluid delivery system.

In alterative variations of the fluid delivery system, the system is configured to deliver potable water. In this embodiment of fluid delivery system, the least one reservoir is configured to store potable water, with the at least one ejector a drinking water ejector disposed in the interior of the vehicle and adapted to eject potable water. The system further includes at least one drinking water valve arranged in fluid communication with at least one pump to control a flow of the potable water from the at least one pump to the at least one drinking water ejector.

Where the fluid delivery system includes the ice maker, the ice maker is disposed on an interior passenger compartment and includes a heat exchanger in thermal communication with the output line of the fluid delivery system and a heat pump in communication with the heat exchanger. A nozzle or port disposed on an end of the output line of the fluid delivery system and provides fluid to the ice maker.

The valve or valves are arranged in fluid communication with the at least one pump and adapted to control a flow of the at least one fluid from the at least one pump to the at least one ejector. The system optionally also includes a second valve disposed in fluid communication with flow of the first fluid. In specific variations, the second valve controls flow of a second fluid to the first fluid flow. Optional valves include a metering valve, mixing valve, or solenoid valve. The valves are under control of the controller, such that the controller actives or deactivates the valve or valves to open and close the valves.

The controller of the fluid delivery system is coupled to the at least one pump and the at least one valve, allowing the controller to activate the at least one pump and the at least one valve upon activation of the fluid delivery system by a user.

The fluid delivery system optionally also includes an air dryer. The air dryer includes an air pump disposed in the vehicle and coupled to the controller, wherein the controller activates the air pump upon activation of the fluid delivery system by a user. A channel is disposed in the vehicle and in fluid communication with the air pump and configured to transfer air from the air pump to a dryer exhaust, where the dryer exhaust disposed on an exterior panel of the vehicle, a door pillar, or sill of the vehicle, or an interior panel of the vehicle. Specific variations of the fluid delivery system also include a brush having a plurality of bristles. The brush is disposed on an exterior panel of the vehicle, a door pillar of the vehicle, or an interior panel of the vehicle.

The fluid delivery system optionally includes at least one cooking fuel ejector in fluid communication with a reservoir and adapted to eject alcohol. The cooking fuel ejector includes at least one cooking valve arranged in fluid communication with the at least one pump to control a flow of alcohol from the at least one pump to the at least one cooking fuel ejector.

The fluid delivery system optionally incorporates a water capture system. Embodiments having the water capture system comprise at least one water collection port disposed on an exterior panel of the vehicle or in the motor compartment of the vehicle and a capture collection reservoir in fluid communication with the at least one water collection port, wherein the capture collection reservoir is configured to retain a fluid and has at least one input and at least one output. The system may have multiple collection ports along upper horizontal body panels of the vehicle, allowing for water to pool around the collection ports, followed by capture of the fluid into the water capture system. The system optionally can include a port disposed adjacent to the vehicle air conditioner (HVAC) and configured to collect condensed water from the HVAC system.

Specific embodiments of the fluid delivery system use a security system to control use of the fluid delivery system. The security system optionally comprises a remote reader sensor and a user security device. The remote reader sensor disposed in the vehicle having a radio frequency transmitter and radio frequency receiver or radio frequency transceiver, where the remote reader sensor is coupled to the controller. The user security device comprises at least a radio frequency transmitter having a preset code assigned in the user security device, and is configured to transmit the preset code upon activation. The remote reader sensor is further configured to transmit an activation signal to the controller upon receiving the preset code. In specific variations of the system, a user-activated switch disposed on an interior panel or the vehicle, user-activated switch disposed on a vehicle door pillar, or a user-activated switch disposed on an exterior panel of the vehicle, where the user-activated switch is activated or otherwise operable by the controller upon receipt of the sensor transmits an activation signal.

### BRIEF DESCRIPTION OF THE DRAWINGS

For a fuller understanding of the invention, reference should be made to the following detailed description, taken in connection with the accompanying drawings, in which:

FIG. 1 is an isometric view of the fluid delivery system internal components according to an embodiment of the disclosure.

FIG. 2 is an isometric view of the fluid delivery system internal components according to an embodiment of the disclosure.

FIG. 3 is an isometric view of the fluid delivery system internal components according to an embodiment of the disclosure.

FIG. 4 is a side view of a venturi mixing chamber.

FIG. 5 illustrates a schematic view of a fluid delivery system mounted on a vehicle and having a plurality of discharge systems, according to an embodiment of the disclosure.

FIG. 6 is a side view of a vehicle depicting a foot wash ejector of a foot wash system of the fluid delivery system, according to an embodiment of the disclosure.

FIG. 7 is an isometric view of a vehicle depicting a user washing system and arranged at an extended position, according to an embodiment of the disclosure.

FIG. 8 is an isometric view of a vehicle depicting a defroster ejector of an defroster system and arranged at an extended position, according to an embodiment of the disclosure.

FIG. 9 is an isometric view of a vehicle depicting the defroster ejector of FIG. 8 and arranged at a retracted position, according to an embodiment of the disclosure.

FIG. 10 is an isometric view of a vehicle depicting a plurality of emitter ports as the plurality of defroster/deicer ejectors, according to an embodiment of the disclosure.

FIG. 11 is an isometric view of a vehicle depicting harm deterrent ejectors of a harm deterrent system of the fluid delivery system, according to an embodiment of the disclosure.

FIG. 12 is an isometric view of a plurality of fire deterrent ejectors of a fire suppression system of the fluid delivery system, according to an embodiment of the disclosure.

FIG. 13a is an isometric view of a fire deterrent ejector of the fire suppression system arranged inside a compartment housing a power source of the vehicle, according to an embodiment of the disclosure.

FIG. 13b is an isometric view of a fire deterrent ejector of the fire suppression system arranged inside a compartment housing a power source of the vehicle, according to an embodiment of the disclosure.

FIG. 14 is an isometric view of vehicle wash ejectors of a vehicle wash system of the fluid delivery system and mounted on a front of the vehicle, according to an embodiment of the disclosure.

FIG. 15 is an isometric view of the fluid delivery system used for hydroponics, aeroponics, and/or aquaponics.

FIG. 16 is an isometric view of a cooking system of the fluid delivery system having a cooking stove arranged inside a storage compartment of the vehicle, according to an embodiment of the disclosure.

FIG. 17 is a plan view of a vehicle dashboard with an integrated drinking water dispensing system, according to an embodiment of the disclosure.

FIG. 18 is a diagram of an electrolysis unit of an electrolytic system, according to an embodiment of the disclosure.

FIG. 19 is a schematic of the fluid delivery system, according to an embodiment of the disclosure.

### DETAILED DESCRIPTION OF THE PREFERRED EMBODIMENT

The inventive fluid delivery system is designed to be mounted on vehicle 200. Fluid delivery system 100 is formed of at least a first reservoir 102 at least a first pump 110 fluidly connected to at least one fluid ejector.

As used herein, “about” means approximately or nearly and in the context of a numerical value or range set forth means±15% of the numerical.

As used herein, “disposed along” means that the structure either directly or indirectly contacts the surface of another structure, while not physically attached to the other structure.

As used herein, “ejector” means a device that directs the flow of a fluid, such as a pressurized fluid, to remove the fluid from a region of higher pressure to a region of lower pressure.

As used herein, “line” means piping for conveying a fluid.

As used herein, “nozzle” means a projecting spout, terminal discharging pipe, or the like, at the end of a pipe, hose, or tube used to control a jet of gas or liquid.

As used herein, “port” means an opening in the face of a passageway for exhaust of a fluid.

As used herein, “pump” means a mechanical device using pressure to move the liquid to another location.

As used herein, “reservoir” means a storage container, containing a fluid.

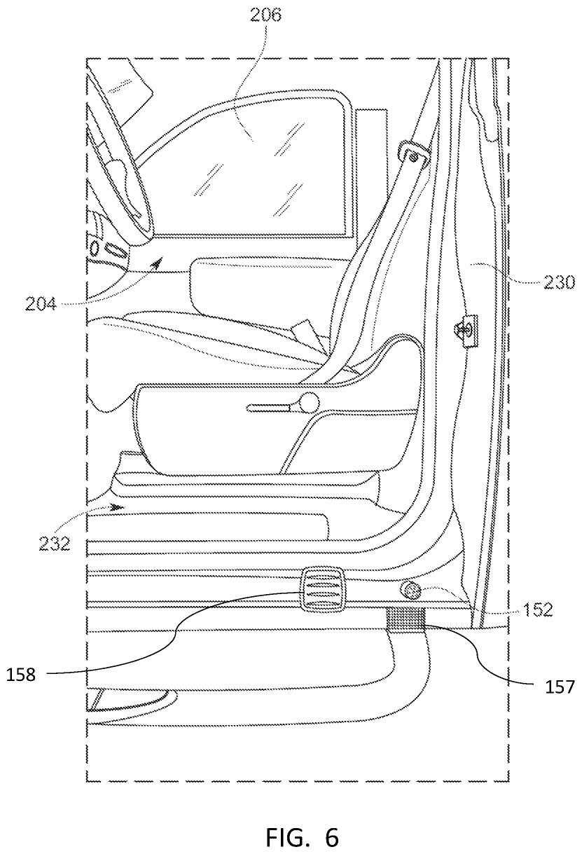

As used herein, “upper” and “lower” reference the orientation shown in FIG. 6. “Upper” is in a direction toward the top of FIG. 6, and “lower” or “bottom” is directed toward the bottom of FIG. 6.

As used herein, “substantially” means largely if not wholly that which is specified but so close that the difference is insignificant.

Where thickness is used herein, such numerical values reflect the outermost diameter of the tubing or other material.

### Example 1

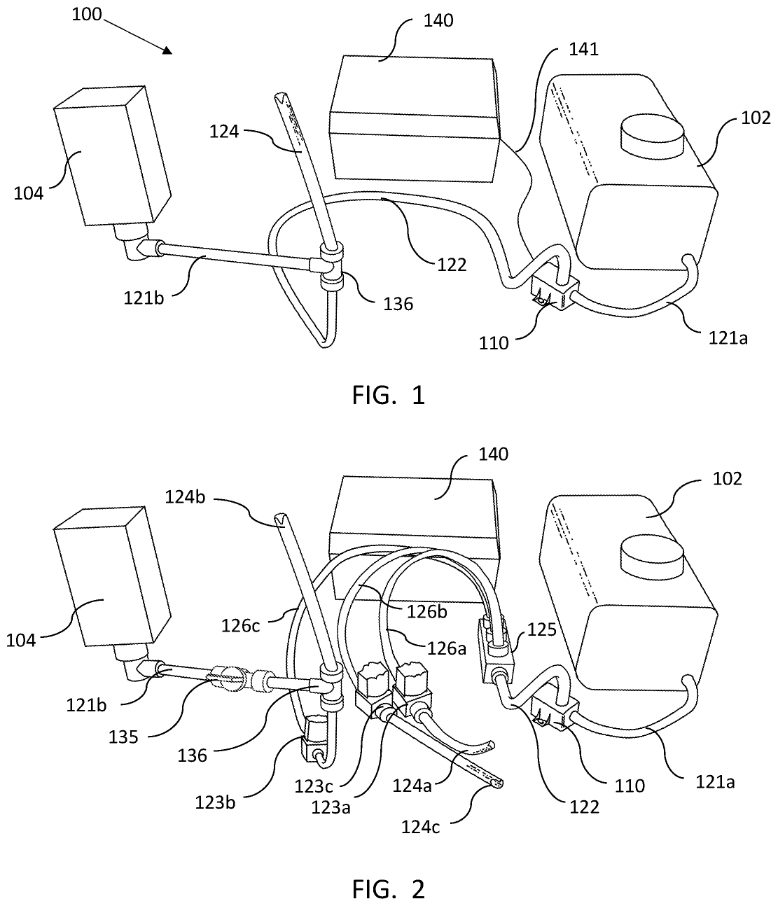

Fluid delivery system 100 is formed of two or more reservoirs, connected to a pump, and adapted to be mounted on vehicle 200. Fluid delivery system 100 is composed of the first reservoir 102, having an input aperture and output aperture, as seen in FIG. 1. First reservoir output line 121a is connected to the output aperture of first reservoir 102 on a first end and pump 110 input on a second end. The output of pump 110 is connected to pump output line 122. Pump output line 122 includes mixing valve 136 or venturi mixing chamber 136b along its length. Second reservoir 104 possesses an output aperture, whereby the output aperture is connected to second reservoir output line 121b on a first end of the line and whereby mixing valve 136 or venturi mixing chamber 136b is connected to the second end of second reservoir output line 121b. Delivery output line 124 connects to mixing valve 136, which runs to the first ejection port, such as hand wash ejector 132.

Controller 140 is in electrical communication with pump 110 using electrical line 141. A user activates fluid delivery system 100 through a switch, or trigger, or activator on an ejection port, such as those discussed in Example 6 through 20, or a combination thereof. Activation of the trigger on an ejection port sends an electrical signal to controller 140, thereby activating fluid delivery system 100. Controller 140 sends an electrical signal to pump 110, resulting in fluid being drawn from first reservoir 102, and pumping the first fluid through pump output line 122. As first fluid traverses mixing valve 136 or venturi mixing chamber 136b, a second fluid is drawn from second reservoir 104 and first fluid and second fluid mixed.

### Example 2

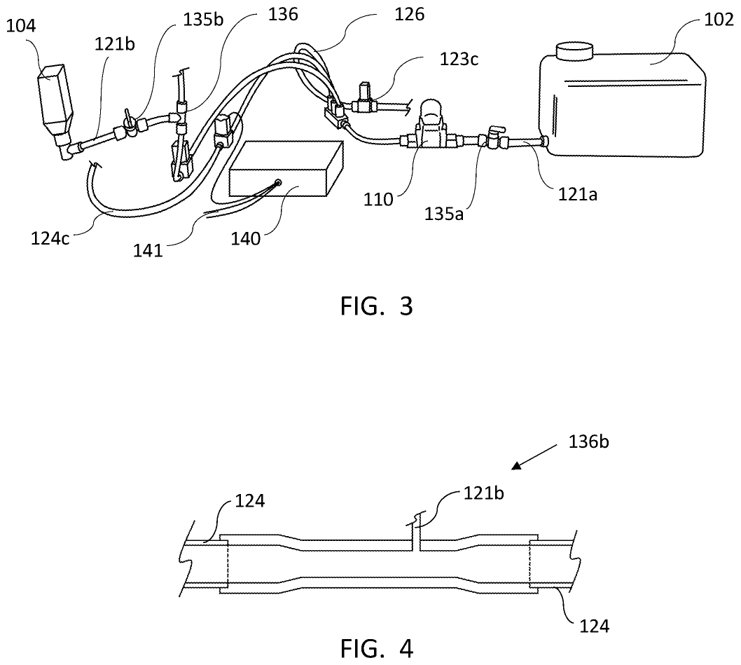

Fluid delivery system 100 is composed of first reservoir 102, as described in Example 1. First reservoir output line 121a is connected to the output aperture of first reservoir 102 on a first end and first pump 110 input on a second end, as seen in FIG. 2. The output of first pump 110 is connected to pump output line 122, whereby pump output line 122 connects to manifold 125, permitting the first fluid to traverse different pathways in fluid delivery system 100. Manifold 125 is shown having three outputs, however, the manifold may possess two or more outputs as needed for the system to operate as desired. First output manifold line 126a exists a first output on manifold 125 and travels along a first pathway to first solenoid valve 123a, whereas second output manifold line 126b exists a second output on manifold 125 and travels along to second solenoid valve 123b, and third output manifold line 126c exists a third output on manifold 125 and travels to third solenoid valve 123b. First delivery output line 124a connects to first solenoid valve 123a on a first end and a fluid ejector on a second end. Second delivery output line 124b connects to second solenoid valve 123b on a first end and adjoins with mixing valve 136 or venturi mixing chamber 136b, terminating in a fluid ejector on a second end, such as hand wash ejector 132. In the depicted embodiment, third delivery output line 124c connects to third solenoid valve 123c on a first end and a fluid ejector on a second end. However, more than one line can be connected to second reservoir 104.

Second reservoir 104 possesses an output aperture, whereby the output aperture is connected to second reservoir output line 121b on a first end of the line and whereby mixing valve 136 or venturi mixing chamber 136b is connected to the second end of second reservoir output line 121b. Metering valve 135 can be disposed on second reservoir output line 121b to control amounts of second fluid flowing into second delivery output line 124b and mixing with the first fluid.

Controller 140 is in electrical communication with pump 110 and of first solenoid valve 123a, second solenoid valve 123b, and third solenoid valve 123c. A user can activate fluid delivery system 100 through a switch, or trigger, or activator on an ejection port. Activating a trigger on an ejection port sends an electrical signal to controller 140, thereby activating fluid delivery system 100. Controller 140 sends an electrical signal to pump 110, resulting in fluid being drawn from first reservoir 102. Concurrently, controller 140 activates mixing valve 136, resulting in fluid in second reservoir 104 flowing into pump output line 122 and mixing with the fluid from first reservoir 102. Where venturi mixing chamber 136b is used in place of mixing valve 136, controller 140 bypasses this step, and fluid flowing from pump 110 draws fluid from second reservoir 104 and concurrently mixes the two fluids. Controller 140 also activates the appropriate solenoid valve, sending the mixed solution to the ejection tube activated by the user.

### Example 3

Fluid delivery system 100 is composed of first reservoir 102, second reservoir 104, pump 110, manifold 125, and the solenoids, as discussed in Example 2. In the present embodiment, first reservoir output line 121a includes first fluid metering valve 135a disposed in line between first reservoir 102 and pump 110. Second reservoir output line 121b includes second fluid metering valve 135b, and mixing valve 136.

The output of first pump 110 is connected to pump output line 122, whereby pump output line 122 connects to manifold 125, permitting the first fluid to traverse different pathways in fluid delivery system 100. Manifold 125 is shown having three outputs, however, the manifold may possess two or more outputs as needed for the system to operate as desired. First output manifold line 126a and third output manifold line 126c are similar to those described in Example 2. Second output manifold line 126b exists a second output on manifold 125 and travels along a second pathway to second solenoid valve 123b. After second solenoid valve 123b, second output on manifold 125b joins with mixing valve 136. Controller 140 comprises circuitry to control pump 110 and the solenoid valves.

### Example 4

Venturi mixing chamber 136b is connected to second delivery output line 124b and is in line with the output line, i.e. disposed to maintain laminar flow in the second delivery output line. Venturi mixing chamber 136b narrows in a center section to form a venturi jet, resulting in an increase in flow of the first fluid through the venturi mixing chamber. Second reservoir output line 121b terminates in the side of Venturi mixing chamber 136b, within the venturi jet, such that flow of the first fluid draws the second fluid into the mixing chamber.

### Example 5

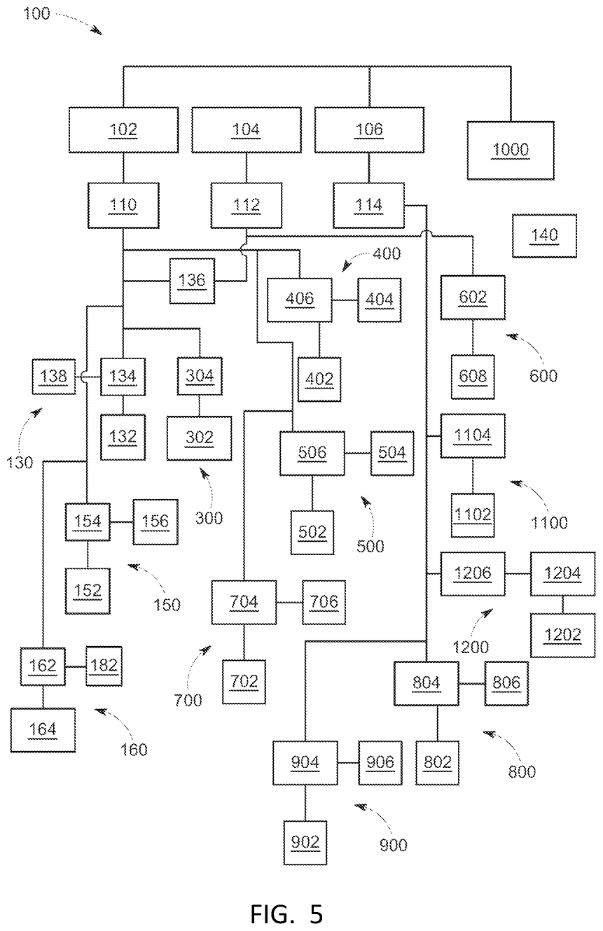

Fluid delivery system 100 is shown in a schematic, as seen in FIG. 5, and includes a fluid delivery system. In this embodiment, fluid delivery system is composed of first reservoir 102, second reservoir 104, and third reservoir 106, to store a first fluid, a second fluid, and a third fluid, respectively. In some variations, first reservoir 102 may be adapted to store non-potable water, second reservoir 104 adapted to store a disinfecting liquid, for example, alcohol, and a third reservoir 106 to store potable water. Fluid delivery system may include water collection system 1000, fluidly connected to first reservoir 102 or first reservoir 102 and third reservoir 106. Where water collection system 1000 is connected to a potable water reservoir, a filter is disposed on the connection between the water collection system and the reservoir, where the filter is adapted to extract microbes and particulates from the water.

The disclosed embodiment depicts first pump 110 disposed in fluid communication with first reservoir 102, second pump 112 disposed in fluid communication with second reservoir 104, and third pump 114 in fluid communication with third reservoir 106. The fluid delivery system 100 is configured to provide the potable water to one or more locations/stations, the non-potable water to one or more locations/stations, the disinfecting liquid to one or more locations/stations, and/or a mixture of the disinfecting liquid and the non-potable/potable water to one or more locations/stations. For illustration purposes, first reservoir 102 and first pump 110 are connected to one or more discharge systems of the fluid delivery system 100. As shown in FIG. 5, first reservoir 102 and first pump 110 are directly connected to fire suppression system 400, and rinse system 700. First reservoir 102 and first pump 110 adjoin, with second reservoir 104 and second pump 112, such that fluid from first reservoir 102 and second reservoir 104 mix. The mixed first fluid and second fluid are connected to hand wash system 130, foot wash system 150, defroster system 160, harm deterrent system 300, and vehicle wash system 500. Second reservoir 104 and second pump 112 are directly connected to cooking system 600. Third reservoir 106 and third pump 114 are directly connected to eye wash system 800, drinking water dispensing system 900, ice maker system 1100, and electrolytic system 1200.

Fire suppression system 400 includes fire suppression valve 406 between fire retardant ejector 402 and the fluid reservoir. Fire suppression sensor 404 is in communication with fire suppression valve 406 and controls activation of fire suppression system 400.

Rinse system 700 includes rinse valve 704 between rinse ejector 702 and the fluid reservoir. Rinse button switch 706 is in communication with rinse valve 704 and controls activation of rinse system 700.

Hand wash system 130 includes hand wash valve 134 and hand wash ejector 132. The hand wash valve is disposed between hand wash ejector 132 and the fluid reservoirs. Hand wash switch 138 is in communication with hand wash valve 134 and controls activation of hand wash system 130.

Foot wash system 150 includes foot wash valve 154 under control of foot wash switch 156, and foot wash ejector 152.

Defroster system 160 includes defroster valve 162 between defroster ejector 164 and the fluid reservoirs. Defroster button/switch 182 is in communication with defroster valve 162 and controls activation of defroster system 160.

Harm deterrent system 300 includes harm deterrent valve 304 between harm deterrent ejector 302 and the fluid reservoirs.

Vehicle wash system 500 includes vehicle wash valve 506 between vehicle wash ejector 502 and the fluid reservoirs. Vehicle wash valve 506 is in communication with vehicle wash switch 504 and under control of the switch, which controls activation of vehicle wash system 500.

Cooking system 600 includes cooking valve 602 between cooking fuel ejector 608 and the fuel source reservoir. In the disclosed embodiment, the fuel source is the second fluid, which acts as a detergent and fuel source, such as isopropyl alcohol. Cooking valve 602 is accessible to a user and controls activation of cooking system 600.

Eye wash system 800 includes eye wash valve 804 between eye wash ejector 802 and third fluid reservoir. In the disclosed embodiment, the third fluid is potable water or other sterile fluid. Eye wash switch 806 is in communication with eye wash valve 804 and controls activation of eye wash system 800.

Drinking water dispensing system 900 includes drinking water ejector 902 and drinking water valve 904 disposed between the ejector and the fluid reservoir. In this embodiment, the fluid is potable water. Drinking button/switch 906 is in communication with drinking water valve 904 and controls activation of drinking water dispensing system 900.

Ice maker system 1100 includes ice maker 1102 and ice making valve 1104. The system optionally includes a valve integrated in the ice maker, in communication with controller 140.

Electrolytic system 1200 includes electrolysis unit 1202 and electrolytic ejector 1204 under control of electrolytic control valve 1206.

Controller 140 is in electrical communication with the actuating switches and valves, as well as the pumps and valves controlling fluid flow, thereby allowing the controller to direct fluid flow through the system. The controller includes hardware and/or software to permit the controller to activate and deactivate the system as needed, such as when a user depresses a switch or after a predetermined period of time. Such systems can include flash hard drives, solid state hard drives, receivers, transmitters, or transceivers to activate and deactivate the system.

### Example 6

Fluid delivery system 100, as described in Example 1, 2, 3 or 5, is disposed in vehicle 200, and includes foot wash system 150. Pump output line 122 is disposed within vehicle 200, terminating in foot wash ejector 152. Vehicle 200 includes door opening 232 associated with door 206 and door sill 230. Foot wash ejector 152 is disposed in the door sill, on a lower face of body panel 230, as seen in FIG. 6, and directed downward toward the ground. However, foot wash system 150 may be installed at various locations of vehicle 200, i.e. foot wash ejectors 152 may be located anywhere on vehicle 200.

Foot wash ejector 152 is in fluid communication with the internal components of fluid delivery system 100, including first fluid reservoir 102 and pump 110, and second reservoir 104, such that foot wash ejector 152 discharges a mixture of water and disinfecting liquid upon activation of the foot wash system, to facilitate a washing and sterilizing of feet of an entrant or occupant of vehicle 200. Foot wash system 150 is under control of foot wash switch 156, which acts as an activator for the system. Accordingly, foot wash switch 156 is in communication with controller 140. In an embodiment, foot wash switch 156 is a sensor mounted at a location proximate to the location of the foot wash ejector, and adapted to detect a presence of an object in the vicinity (i.e., below the foot wash ejector 152) of the foot wash ejector.

Foot wash system 150 may include at least one foot wash valve 154 to control a flow of the mixture of the water and the disinfecting liquid to the at least one foot wash ejector 152. Useful foot wash valve 154 include an electrically actuated valve adapted to control a flow of the mixture of water and disinfecting liquid to the connected foot wash ejector 152. The foot wash valve may be directly under control of foot wash switch 156 or may be controlled by controller 140.

It may be appreciated that one foot wash switch 156 is associated with one foot wash valve 154 and a single foot wash ejector 152 or nozzle. As a user or entrant activates foot wash switch 156, a signal is transmitted to the controller or the controller and the associated foot wash valve 156, resulting in the associated foot wash valve being actuated and causing a flow of the mixture of the water and disinfecting liquid to the associated foot wash ejector.

In an embodiment, brush 157 is disposed adjacent to foot wash ejector 152, permitting a user to remove loose debris from the user's foot or footwear. The depicted embodiment also shows air blower 158 disposed adjacent to foot wash ejector 152. Air blower 158 is either in communication with controller 140, and activated concurrently with foot wash ejector 152 or at a predetermined delay. Controller 140 deactivates foot wash ejector 152 followed by air blower 158 at a predetermined time after foot wash ejector 152 is deactivated. Alternatively, a sensor may be mounted at a location proximate to air blower 158 to detect a presence of an object in the vicinity (i.e., below air blower 158), and the controller 140, upon detection of the presence of the object, actuates the associated air blower 158 to allow the flow air to dry the user's foot or footwear.

Foot wash system 150 may be actuated upon detection of an opening of the door 206 of the passenger compartment 204. It may be envisioned that a pressure sensor, light sensor, motion sensor, or other system known to one of skill in the art may be used to actuate the foot wash system upon opening of a vehicle door. Furthermore, sensors can also be used to disable the system when the vehicle is in motion, such as by linking controller 140 to the vehicle onboard computer. Alternatively, a button adapted for a user to depress or a sensor disposed adjacent to the foot wash ejector 152 may be used to actuate the system. Foot wash valve 154 may be turned off automatically after an elapse of a predetermined duration from the actuation. It may be appreciated that the sensors are in communication with controller 140, whereby controller 140 activates any pumps and valves associated with foot wash system 150, such as pump 110 or first pump 110 and second pump 112, mixing valve 136 and metering valve 135/second fluid metering valve 135b. This facilitates priming the fluids and transferring the fluids to allow a flow of the disinfecting liquid to foot wash ejector 152. Also, mixing valve 136 is turned off automatically along with the foot wash valve 154. Specific embodiments of the foot wash system 150 include one or more lights, for example, light emitting diodes, directed to permit a user to visualize the cleaning process, such as disposed on or adjacent to the foot wash ejector.

### Example 7

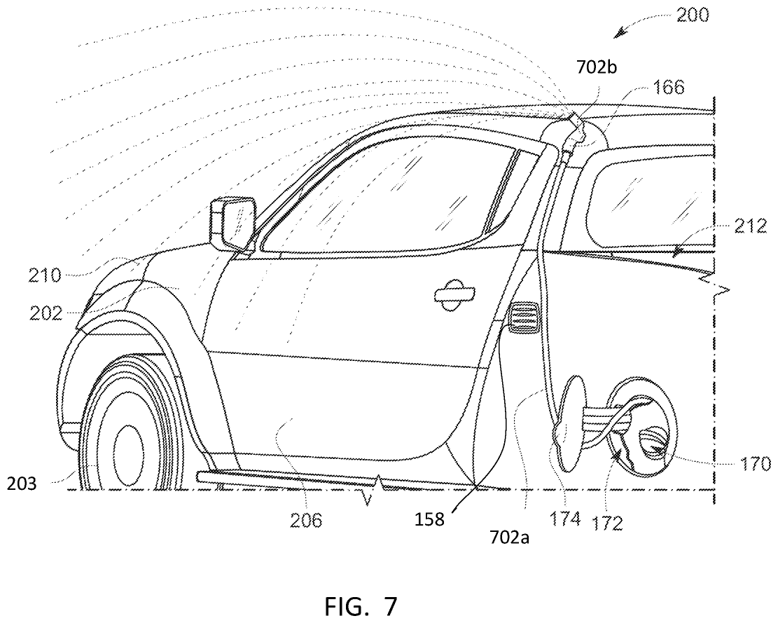

Fluid delivery system 100 as described in Example 1, 2, 3, or 5 is disposed in vehicle 200, and includes rinse system 700. Pump output line 122 is disposed within vehicle 200, terminating in rinse ejector 702. Vehicle 200 includes body panels associated with vehicle body 202, whereby rinse ejector 702 is disposed on a body panel or within a compartment on the body panel, such as fuel inlet port 170 or charging port, as seen in FIG. 7. Rinse ejector 702 is a shower head in the depicted embodiment, and includes rinse ejector line 702a and rinse ejector head 702b. Rinse button switch 706 is disposed on rinse ejector head 702b in the depicted embodiment, and in communication with rinse valve 704 and controls activation of rinse system 700. However, rinse ejector head 702 may also be a port or nozzle. As such, rinse system 700 may be used as a cloth wash/rinsing system or a body wash system, or a vehicle body wash system.

Rinse ejector 702 is in fluid communication with the internal components of fluid delivery system 100, including first fluid reservoir 102 and pump 110, and optionally, second reservoir 104, allowing rinse ejector 702 to discharge non-potable water or a mixture of water and disinfecting liquid. Rinse system 700 is under control of rinse button/switch 706, which is in communication with controller 140 and activates pump 110, pump 110 and solenoid valve 123. Rinse system 700 may include at least one rinse valve 704 to control a flow of water, or the mixture of the water and the disinfecting liquid to the rinse ejector. Useful valves include an electrically actuated valve adapted to control a flow of water or the mixture of water and disinfecting liquid to the connected rinse ejector 702. The rinse valve may be directly under control of rinse button switch 706 or may be controlled by controller 140.

In an embodiment, air blower 158 is disposed on a body panel adjacent or near to rinse ejector 702. Air blower 158 is either in communication with controller 140, and activated concurrently with rinse ejector 702 or at a predetermined delay. Controller 140 deactivates rinse ejector 702 followed by air blower 158 at a predetermined time after rinse ejector 702 is deactivated, such as when the user deactivates rinse button switch 706. Alternatively, a sensor may be mounted at a location proximate to air blower 158 to detect a presence of an object in the vicinity (i.e., below air blower 158), and the controller 140, upon detection of the presence of the object, actuates the associated air blower 158 to allow the flow air to dry the user.

### Example 8

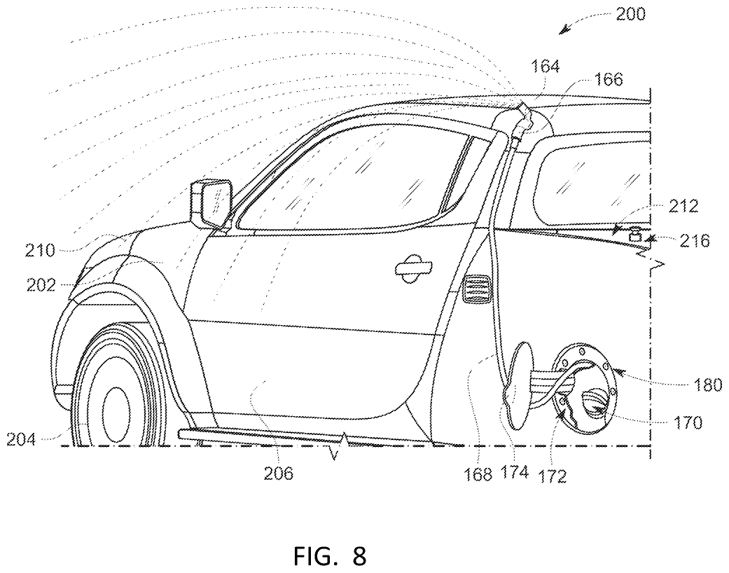

Fluid delivery system 100 as described in Example 1, 2, 3, or 5 is disposed in vehicle 200, and includes defroster system 160. Pump output line 122 is disposed within vehicle 200, terminating in one or more defroster ejectors 164 (i.e., emitter ports and/or nozzles) arranged at various locations around vehicle body 202 to eject the mixture of the disinfecting liquid and water. Defroster system 160 includes defroster ports and/or nozzles, such as nozzle 166 connected to a hose 168 that can be extended or retracted relative to the vehicle body 202 to assist in defrosting/deicing a vehicle. Defroster ejectors 164 may be disposed along the body panels associated with vehicle body 202, and/or disposed within a compartment on the body panel, such as fuel inlet port 170 or charging port, as seen in FIG. 8. Activation of defroster system 160 occurs via actuating defroster button/switch 182 provided on the nozzle 166, on an interior panel or on a user transmitter, and is in communication with controller 140.

Defroster ejectors 164 is composed of nozzle 166 or nozzle 166 is a single component of the defroster system 160. Nozzle 166 is arranged proximate to fuel inlet port 170 or charging port and is arranged inside a compartment 172 that is accessed by opening a gate 174. The nozzle 166 may be manually extended after opening the gate 174. When a handle of the nozzle 166 is pressed, the mixture of the water and the disinfecting liquid from the nozzle 166 is ejected. By extending the nozzle 166, the mixture of disinfecting liquid and the water can be sprayed at the desired locations to melt the ice. Accordingly, the handle and the nozzle 166 together acts as the ice melting valve 162. Upon actuation by defroster button/switch 182, controller 140 activates pump 110 and solenoid valve 123. Defroster button/switch 182 triggers ice melting valve 162 open to allow the flow of mixture of the disinfecting liquid and water from the fluid reservoirs to emitter ports 180. Ice melting valve 162 is shut-off either after an elapse of a certain time duration from the actuation of the ice melting valve 162 or by activating the switch 182 again. In an embodiment, the ice melting system 160 may include at least one temperature sensor to monitor ambient temperature, whereby controller 140 actuates ice melting valve 162 to deliver the mixture of water and disinfecting liquid based on the ambient temperature and time duration elapsed since turning off the vehicle 200.

The input to discharge the fluid through the emitter ports 180 may be provided by actuating defroster button/switch 182, provided on a key of vehicle 200, disposed inside vehicle 200, or a virtual switch provided on a smartphone application. In some embodiments, the virtual switch 182 may be activated by the voice command. In some embodiments, the defroster ports 180 may also be located along the opening of the fuel inlet compartment 172.

### Example 9

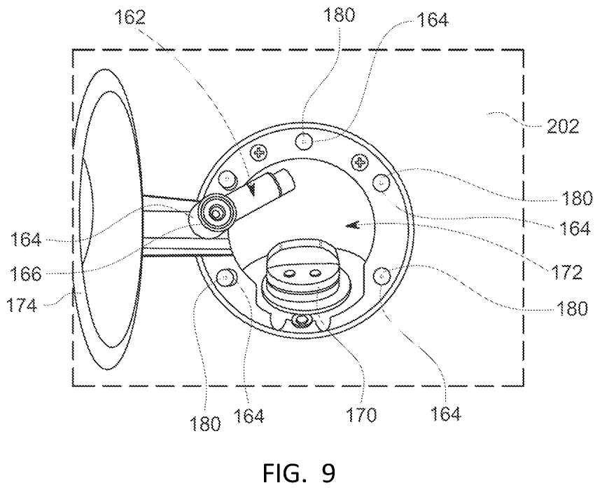

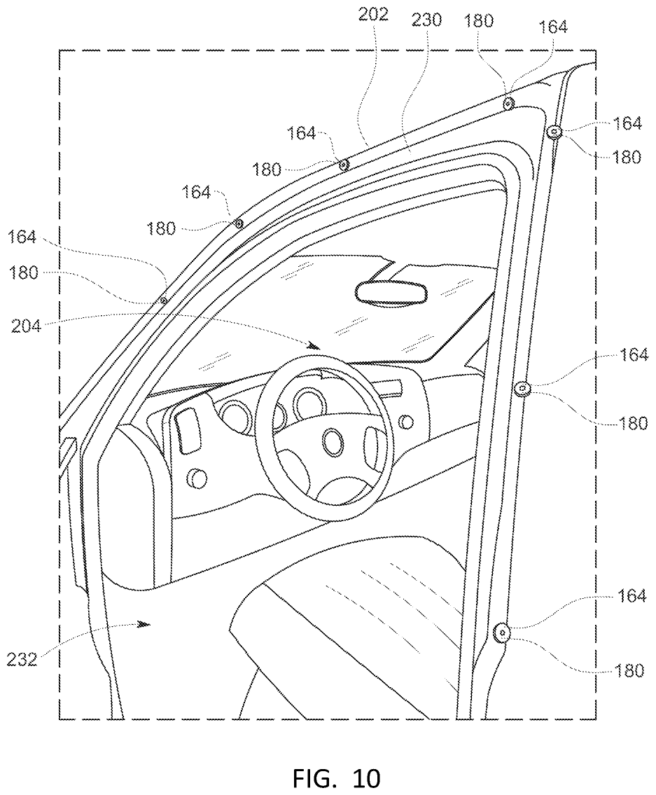

Fluid delivery system 100 as described in Example 1, 2, 3, or 5 is disposed in vehicle 200, and includes defroster system 160 in fluid communication with the internal components of fluid delivery system 100, including first fluid reservoir 102 and pump 110, and second reservoir 104, such that defroster system 160 discharges a mixture of water and disinfecting liquid upon activation of the system. Pump output line 122 is disposed within vehicle 200, terminating in one or more defroster ejectors 164 (i.e., emitter ports and/or spray heads) arranged at various locations around vehicle body 202 to eject the mixture of the disinfecting liquid and water. Defroster system 160 includes defroster ports 180 disposed at various sections of the vehicle, such as at the opening of the fuel inlet compartment 172, as seen in FIG. 9. Additionally, defroster ports 180 are disposed the along body panels 230 of door openings 232 associated with doors 206 of the vehicle 200, as seen in FIG. 10. Activation of defroster system 160 occurs via actuating defroster button/switch 182 provided on the nozzle 166, on an interior panel or on a user transmitter, and is in communication with controller 140.

The input to discharge the fluid through the emitter ports 180 may be provided by actuating defroster button/switch 182, provided on a key of vehicle 200, disposed inside vehicle 200, or a virtual switch provided on a smartphone application. In some embodiments, the virtual switch 182 may be activated by the voice command.

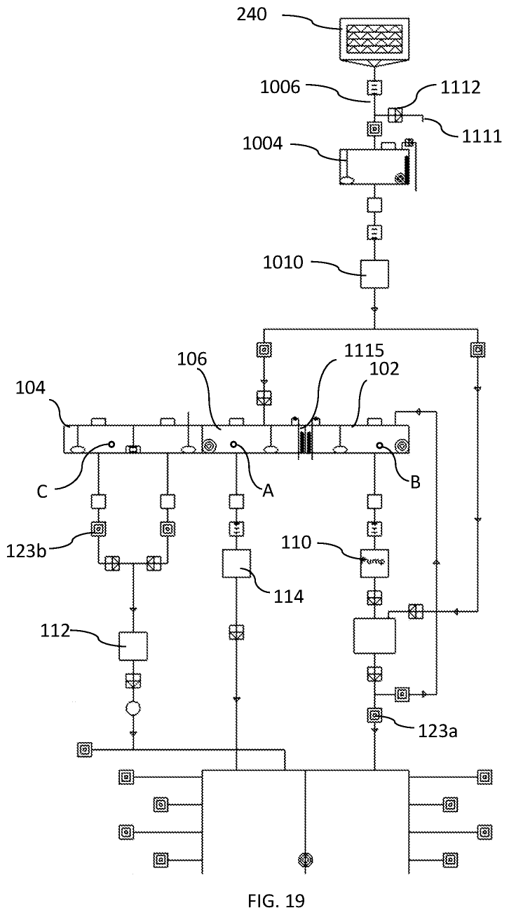

Upon actuation by defroster button/switch 182, controller 140 activates pump 110 and solenoid valve 123. Defroster button/switch 182 triggers defroster valve 162 open to allow the flow of mixture of the disinfecting liquid and water from the fluid reservoirs to emitter ports 180. Defroster valve 162 is shut-off either after an elapse of a certain time duration from the actuation of the defroster valve 162 or by activating the switch 182 again. In an embodiment, the defroster system 160 may include at least one temperature sensor to monitor ambient temperature, whereby controller 140 actuates defroster valve 162 to deliver the mixture of water and disinfecting liquid based on the ambient temperature and time duration elapsed since turning off the vehicle 200. Optionally, fluid delivery system 100 includes an electric heater, such as an electric heating coil disposed in or around first fluid reservoir 102 to prevent freezing of the first fluid, such as those depicted in FIG. 19. In specific variations, the second fluid is a disinfecting fluid having a low freezing temperature, such as isopropyl alcohol.

### Example 10

Fluid delivery system 100 as described in Example 1, 2, 3, or 5 is disposed in vehicle 200, and includes hand wash system 130 having at least one hand wash ejector 132 arranged at various locations of vehicle 200 and adapted to discharge water and/or the mixture of water and the disinfecting liquid to facilitate a washing and sterilizing of hands of an entrant or occupant of the vehicle 200. Hand wash system 130 at least one hand wash ejector 132 disposed along vehicle body 202, such as inside compartment 172 that is accessed by opening gate 174 and/or inside a fuel inlet compartment or a charging compartment. However, the hand wash ejector 132 may be located at any location of vehicle 200 and may be arranged on any exterior surface of vehicle 200. Hand wash ejector 132 is optionally a nozzle or port, where a nozzle version may be manually extended after opening gate 174. At least one hand wash valve 134 is provided to control a flow of the mixture of the water and the disinfecting liquid to the at least one hand wash ejector 132.

In an embodiment, each hand wash ejector 132 is controlled by a single hand wash valve 134, such as an electrically actuated solenoid valve, and is adapted to control a flow of the mixture of water and the disinfecting fluid to the associated hand wash ejector 132. For mixing the water with the disinfecting fluid, a portion of the disinfecting liquid from an outlet of the second pump 112 is diverted to an outlet of the first pump 110. For controlling the diversion of the disinfecting fluid to the outlet of the first pump 110, a mixing valve 136 may be provided, according to an embodiment of the disclosure. Alternatively, disinfecting fluid may be drawn into a first fluid, such as water, by the flow of the first fluid.

To actuate the hand wash valve 134, hand wash switch 138 is optionally associated with hand wash valve 134. As a user or entrant activates hand wash switch 138, the switch sends a signal to controller 140, whereby controller 140 activates first pump 110 and second pump 112 and opens hand wash valve 134 associated with the hand wash ejector, and mixing valve 138. Alternatively, controller 140 activates first pump 110 and opens hand wash valve 134 associated with the hand wash ejector, and mixing valve 138, allowing flow of the first fluid to draw the disinfecting fluid into the first fluid and mix the two fluids. In other embodiments, a sensor may be mounted to a location proximate to the location of the hand wash ejector 132 to detect a presence of an object in the vicinity (i.e., below the ejector 132) of the hand wash ejector 132, and the sensor send a signal to controller 140 upon detection of the presence of the object, thereby actuating the pumps and the associated hand wash valve 134 to allow the flow of the mixture of water and disinfecting liquid to the associated hand wash ejector 132. In other embodiments, hand wash system 130 is actuated upon detection of an opening of passenger compartment door 206. A sensor associated with the door, sends a signal to controller 140 on opening the door, thereby actuating the hand wash system. In specific variations of this embodiment, the system is primed but does not eject fluid until the user activates a switch on or adjacent to hand wash ejector 132, limiting unneeded use of fluid from the vehicle.

It may be envisioned that hand wash valve 134 and mixing valve 136 may be turned off (i.e., closed) automatically after an elapse of a predetermined duration from the actuation. In some embodiments, first reservoir 102 stores a mixture of water and disinfecting fluid in a predetermined ratio. In such a case, mixing valve 136 may be omitted. In some embodiments, the at least one hand wash ejector 132 may include one or more lights, for example, light emitting diodes.

### Example 11

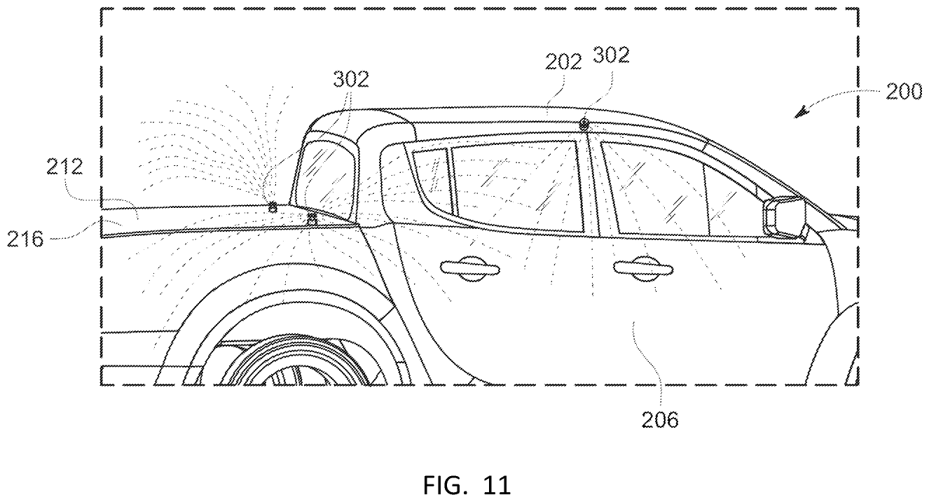

Fluid delivery system 100 as described in Example 1, 2, 3, or 5 is disposed in vehicle 200, and includes harm deterrent system 300 to facilitate a spray of a mixture of disinfecting liquid and water around the vehicle 200 to prevent a theft or a forced opening of the vehicle door 206. The harm deterrent system 300 includes a plurality of harm deterrent ejectors 302 disposed along vehicle body 202, as seen in FIG. 11. Harm deterrent ejector 302 is a port or nozzle connected to at least one harm deterrent valve 304 to facilitate a flow of the mixture of disinfecting liquid and water to the plurality of harm deterrent ejectors 302.

Harm deterrent system 300 is in fluid communication with the internal components of fluid delivery system 100, including first fluid reservoir 102 and pump 110, and second reservoir 104, such that harm deterrent ejector 302 discharges water, or a mixture of water and disinfecting liquid upon activation of the system.

Harm deterrent system 300 is under control of a panic button 308 arranged inside the vehicle 200, which acts as an activator for the system. Accordingly, panic button 308 is in communication with controller 140.

Controller 140 opens the at least one harm deterrent valve 304 and the mixing valve 136, and pump 110 activated to allow the flow of mixture of disinfecting liquid and water to the harm deterrent ejectors 302 in response to an actuation. Harm deterrent system 300 alternatively provides a sensor to detect an attempt of forced opening of door 206, and the controller 140 actuates the harm deterrent valve 304 and the mixing valve 136 to eject the mixture of the alcohol (i.e., disinfecting liquid) and water from the plurality of harm deterrent ejectors 302.

In an embodiment, harm deterrent system 300 is comprised of two systems; one disposed on a first side of vehicle 200 and adapted to eject the mixture of alcohol and water on the first side (left side) of the vehicle, and a second side of vehicle 200 and adapted to eject the mixture of alcohol and water on the second side (right side) of the vehicle. Harm deterrent system 300 thus includes a first harm deterrent valve associated with the first side harm deterrent system to provide the mixture of alcohol and water to the ejectors associated with the first side harm deterrent system; and a second harm deterrent valve associated with the second side harm deterrent system to provide the mixture to the second side ejectors. Further, the harm deterrent system may include separate panic buttons/sensors to actuate the first harm deterrent valve or the second harm deterrent valve independent of the other valve.

Harm deterrent system 300 optionally remains active even after switching off the ignition system of vehicle 200.

Harm deterrent system may use a mixture of isopropyl alcohol and water in a concentrated solution of 60% isopropyl alcohol or above, to facilitate instant discomfort by temporarily disorienting the senses of the nose, mouth, and eyes of a person. This will allow the user some time to escape from a situation. Additionally, harm deterrent ejectors 302 optionally move from a retracted position, when deactivated, to an extended position when the harm deterrent system is activated.

### Example 12

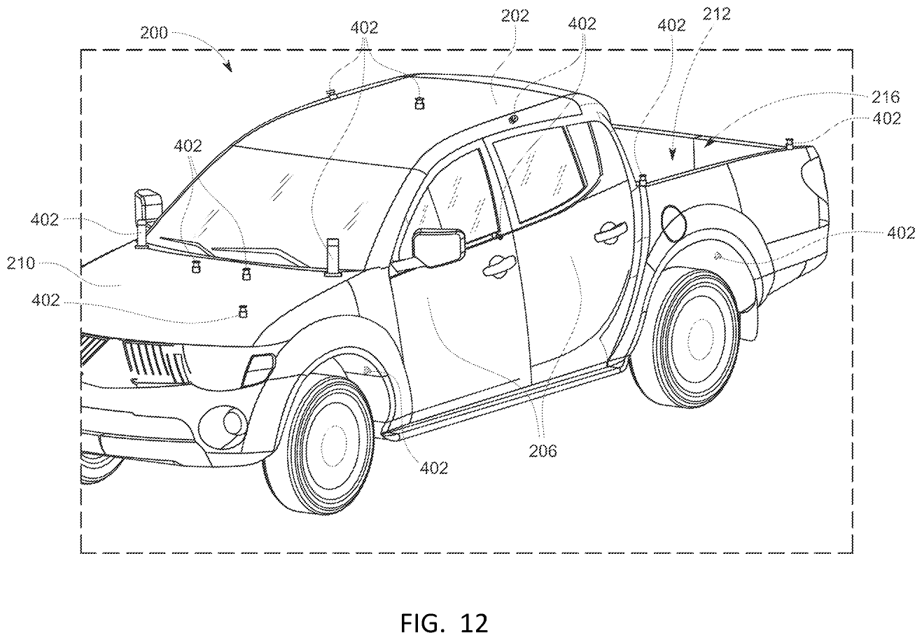

Fluid delivery system 100 as described in Example 1, 2, 3, or 5 is disposed in vehicle 200, and includes fire suppression system 400 to suppress a fire in vehicle 200. The fire suppression system 400 includes at least one fire retardant ejector 402, for example, nozzles/ports, arranged at various locations to spray water, as either a potable water or non-potable water, in response to the detection of fire or smoke at the associated locations on or around the vehicle 200, as seen in FIG. 12.

Fire suppression system 400 is in fluid communication with the internal components of fluid delivery system 100, including first fluid reservoir 102 and pump 110, with the at least one first retardant valve is fluidly connected to the first pump 110 to receive the water from the first reservoir 102.

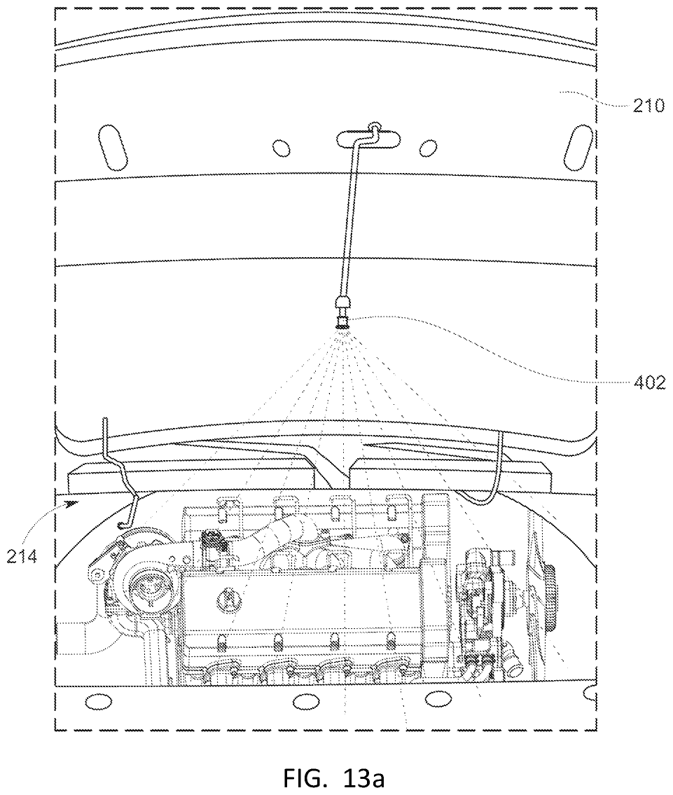

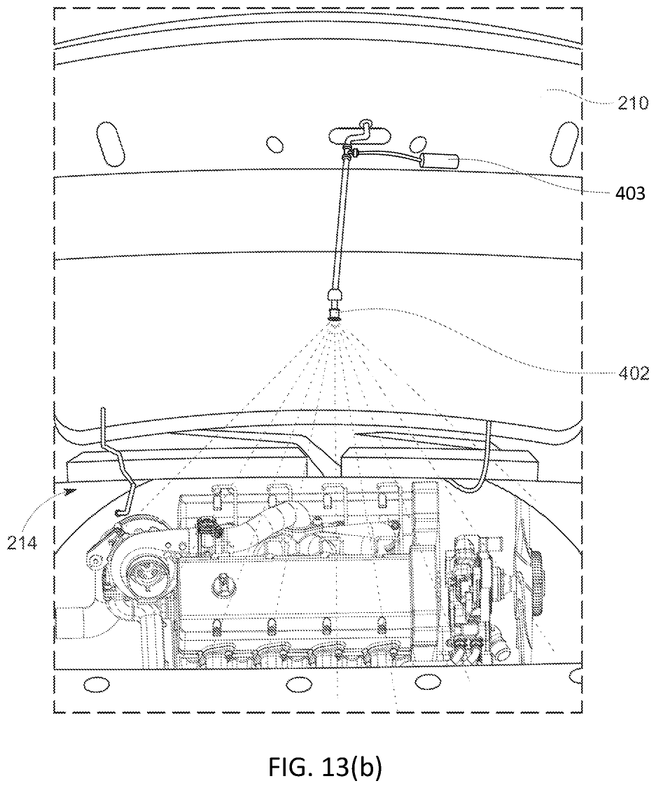

In an example, as shown in FIG. 13a, fire retardant ejector 402 is mounted under the hood 210 inside the compartment 214 to eject water for suppression of the fire or the smoke generated inside the compartment 214. In an embodiment, fire suppression system 400 includes chemical fire retardant 403 mixed in-line with the water prior to being ejected for fire suppression, as seen in FIG. 13b.

For detecting the fire and/or the smoke, the fire suppression system 400 optionally includes at least one sensor 404, for example, at least one fire detection sensor or at least one smoke detection sensor, or at least one thermal detection sensor. Upon detecting the fire or the smoke, the sensor sends a signal to controller 140, whereby controller 140 opens the at least fire retarder valve 406, and pump 110 activated to allow the flow of water to the fire retardant ejectors 402 in response to an actuation. In specific embodiments using multiple fire retarder valves 406, controller 140 actuates the fire retarder valve 406 associated with the sensor, ejecting water from the fire retardant ejectors 402 located at the site of the fire and/or the smoke.

Fire retardant ejectors 402 are optionally retractable nozzles, wherein the nozzles move from a retracted position to an extended position to eject the water upon detection of the fire or the smoke. It may be appreciated that the controller 140 may move fire retardant ejectors 402 to the extended position upon detection of the fire and/or the smoke, and also actuates the at least one retardant valve 406 accordingly.

### Example 13

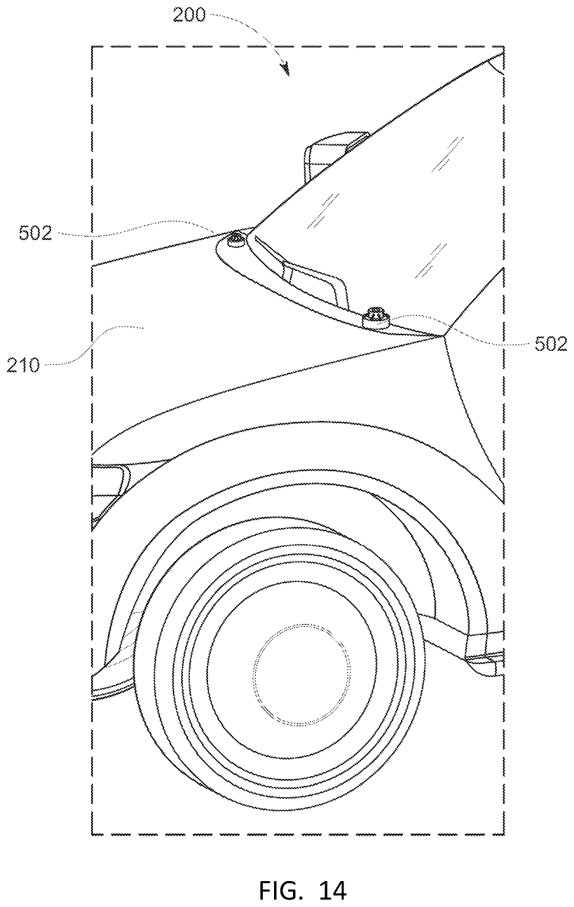

Fluid delivery system 100 as described in Example 1, 2, 3, or 5 is disposed in vehicle 200, and includes vehicle wash system 500, configured to eject water (i.e., non-potable water) on vehicle 200 to clean/wash the vehicle. The vehicle wash system 500 includes at least one vehicle wash ejector 502, as nozzles or pressurized ports, arranged on the vehicle body 202 and adapted to spray water onto the vehicle 200, as seen in FIG. 14. Vehicle wash ejectors 502 are optionally mounted at various locations, for example, front, rear, sides, roof, or hood of vehicle 200. Vehicle wash ejectors 502 may also include a spray head, similar to the defroster ejectors 164 of Example 8.

In an embodiment, the vehicle wash system 500 includes a wash switch 504 and at least one vehicle wash valve 506 in communication with controller 140. Wash system 500 is in fluid communication with the internal components of fluid delivery system 100, including first fluid reservoir 102 and pump 110, such that vehicle wash valve 506 is fluidly connected to the first pump 110 to receive the water from the first reservoir 102.

Wash switch 504 sends a signal to controller 140, to activate first pump 110 and initiate flow of water from first fluid reservoir 102 to vehicle wash ejectors 502. Accordingly, the at least one vehicle wash valve fluidly connects to the fluid lines of the vehicle wash system and controls a flow of water to the at least one vehicle wash ejector. Upon activation of wash switch 504 to a first position, controller 140 opens vehicle wash valve 506, and closes the at least one vehicle wash valve 506 upon movement of the wash switch 504 to a second position or after a predetermined period of time. Accordingly, a user may actuate wash switch 504 to the first position to actuate vehicle wash system 500.

Vehicle wash ejector 502 are optionally nozzles rotatably fixed to the vehicle, thereby allowing the ejector to swivel during spraying. Moreover, the nozzles are also optionally retractable, as discussed above, allowing the ejectors to retract flush with the vehicle body panels when not in use.

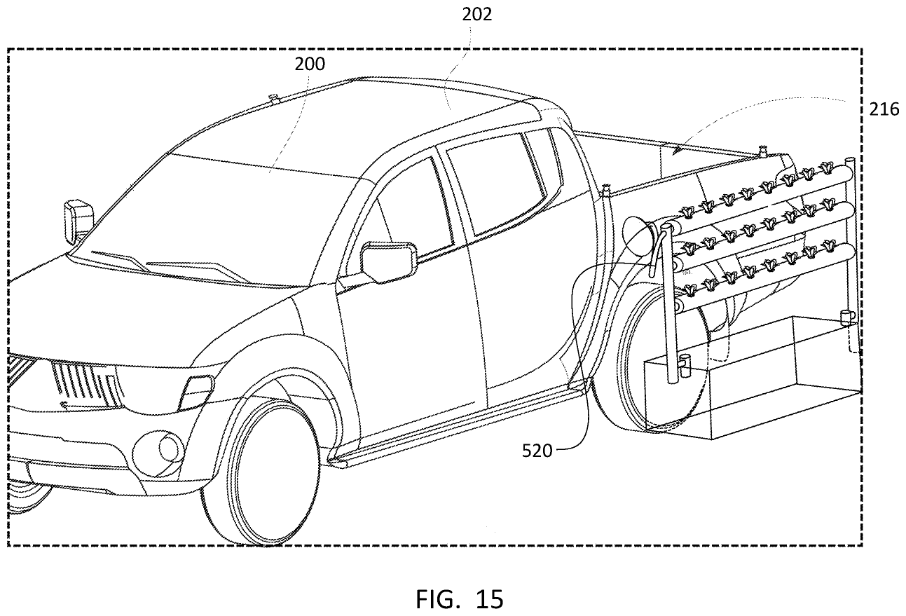

Moreover, the water from first reservoir 102 may be delivered to a hydroponic, and or aeroponic, and or aquaponic growing system to cultivate plants, grow fungi, and farm fish that can be removably engaged to the vehicle, as seen in FIG. 15. In this embodiment, fluid line 520 is connected to the ejector, or the wash ejectors 502 is removed from hose 168, and the hose attached to the hydroponic aeroponic, and or aquaponic growing system.

### Example 14

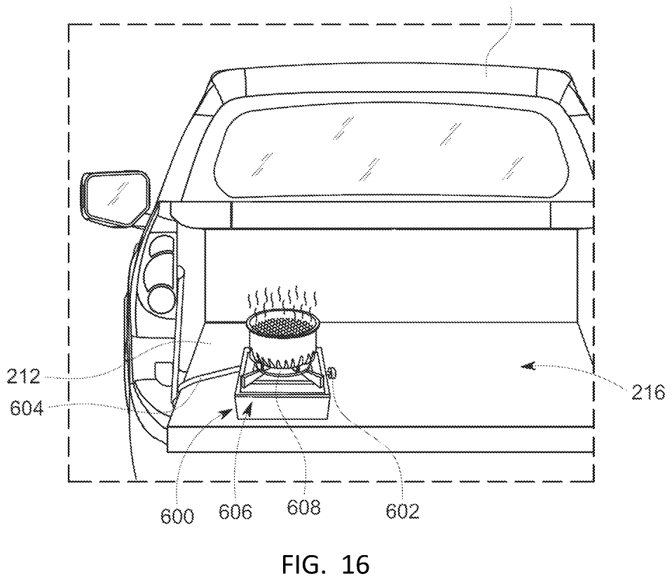

Fluid delivery system 100 as described in Example 1, 2, 3, or 5 is disposed in vehicle 200, and includes cooking system 600, having cooking valve 602, conduit 604, and cooking stove 606 having at least one cooking fuel ejector 608, as seen in FIG. 16.

Cooking system 600 is in fluid communication with the internal components of fluid delivery system 100, including second fluid reservoir 104 and second pump 112, allowing for transfer of a disinfectant fluid that also is flammable. Examples include isopropyl alcohol. Conduit 604 is connected to delivery output line 124, such that disinfectant from second fluid reservoir 104 and second pump 112 may be received by cooking system 600. Cooking valve 602 is fluidly connected with second pump 112 and the cooking stove 606 and controls a flow the alcohol from second pump 112 (i.e., second reservoir 104) is to the at least one cooking fuel ejector 608 of cooking stove 606 to facilitate an initiation of fire for cooking food. Optionally, Cooking valve 602 is in communication with controller 140, providing an activation signal to controller 140 when cooking valve 602 is turned on, resulting in controller 140 activating pump 112.

### Example 15

Fluid delivery system 100 as described in Example 1, 2, 3, or 5 is disposed in vehicle 200, and includes eye wash system 800, to enable washing of the eyes of the user. Eye wash system 800 includes at least one eye wash ejector 802 fluidly connected with the pump to receive fluid from the third reservoir 106, such as potable water, and at least one eye wash valve 804 fluidly connected to delivery output line 124, such as third delivery output line 124c. Where the fluid delivery system is as seen in Example 5, third pump 114 is in fluid communication with eye wash system 800. Optionally the system includes eye wash switch 806, such as a trigger disposed on eye wash ejector 802.

Eye wash valve 804 and eye wash switch 806 are in communication with controller 140, such that the switch sends a signal to controller 140 to activate third pump 114 and initiate flow of water from third fluid reservoir 106 to eye wash ejector 802, while ensuring mixing valve 136 (or any metering or flow valve connected to second fluid reservoir 104) is not active. Accordingly, potable or sterile eye-compatible fluid is transferred from a fluid reservoir to the eye wash ejector. Upon activation of eye wash system, the fluid flows to the at least one eye wash ejector, which is a port or nozzle, to allow a user to wash his or her eye.

### Example 16

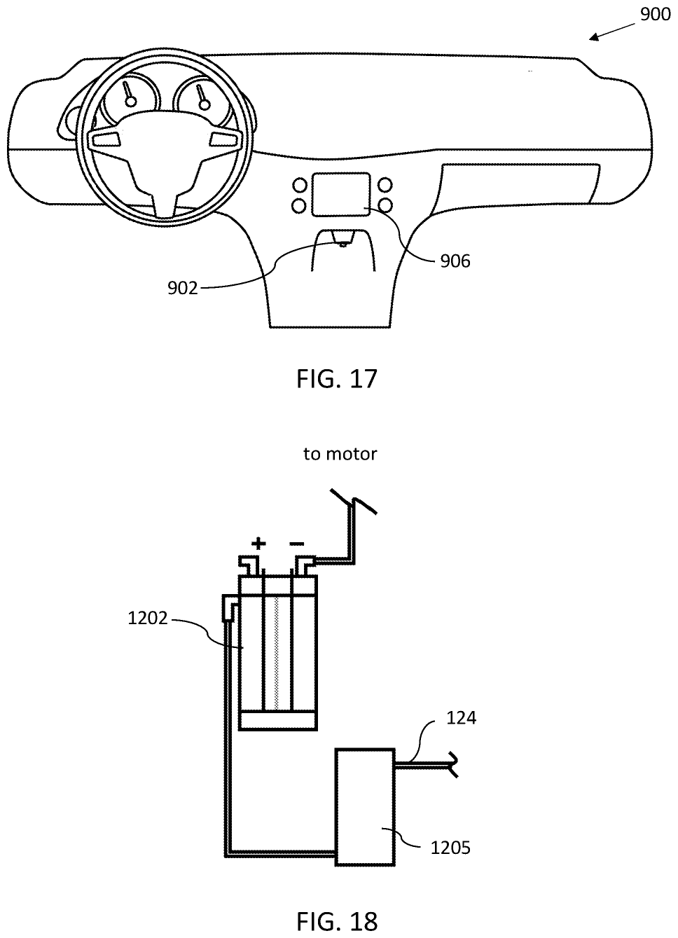

Fluid delivery system 100 as described in Example 1, 2, 3, or 5 is disposed in vehicle 200, and includes drinking water dispensing system 900, to dispense and provide drinking water to the persons present inside vehicle 200. Drinking water dispensing system 900 includes at least one drinking water ejector 902 fluidly connected with the pump to receive fluid from the third reservoir 106, such as potable water, and at least one drinking water valve 904 fluidly connected to delivery output line 124, such as third delivery output line 124c. Where the fluid delivery system is as seen in Example 5, third pump 114 is in fluid communication with drinking water dispensing system 900. Optionally the system includes drink button/switch 906, such as a trigger disposed on or about drinking water ejector 902, as seen in FIG. 17. Drinking water ejector 902 is optionally a port or nozzle directed to permit drinking or filling of a container. In an embodiment, drinking water ejector 902 is arranged proximate to a floor of vehicle 200 to enable drinking of water by a pet.

Drinking water valve 904 or drink button/switch 906 is in communication with controller 140, such that the valve sends a signal to controller 140 to activate third pump 114 and initiate flow of water from third fluid reservoir 106. Alternatively, drinking water valve 904 or drink button/switch 906 sends a signal to controller 140 to activate first pump 110 and initiate flow of potable water from first fluid reservoir 102 to drinking water ejector 902, while ensuring mixing valve 136 (or any metering or flow valve connected to second fluid reservoir 104) is not active. Accordingly, potable fluid is transferred from a fluid reservoir to drinking water ejector 902.

Moreover, the water from third reservoir 106 may be delivered to a hydroponic, and or aeroponic, and or aquaponic growing system to cultivate plants, grow fungi, and farm fish that can be removably engaged to the vehicle.

### Example 17

Fluid delivery system 100 as described in Example 1, 2, 3, or 5 is disposed in vehicle 200, and includes ice maker system 1100, to that facilitates a delivery of the water from the potable reservoir 106 to an ice maker 1102 for making ice.

Ice maker system 1100 includes ice maker 1102 fluidly connected with the pump to receive fluid from the third reservoir 106, such as potable water, and at least one ice making valve 1104 fluidly connected to delivery output line 124, such as third delivery output line 124c. Where the fluid delivery system is as seen in Example 5, third pump 114 is in fluid communication with ice maker system 1100.

Ice maker 1102 receives the potable water from fluid reservoir 106 via at least one ice making valve 1104 in response to an actuation of ice making valve 1104. The fluid delivery system 100 may also provide an evaporative cooling system of the vehicle to evaporatively cool an interior of the vehicle 200 or other living area. As a non-limiting example, a cooling system for vehicle 200 may include a coil in indirect communication with delivery output line 124 or third delivery output line 124c, but concurrently in thermal communication with the output line, allowing the cooling system to cool the potable water.

### Example 18

Fluid delivery system 100 as described in Example 1, 2, 3, or 5 is disposed in vehicle 200, and includes electrolytic system 1200, to convert water received from first reservoir 102 or third reservoir 106 to hydrogen and oxygen.

Electrolytic system 1200 has electrolysis unit 1202 fluidly connected with electrolytic ejector 1204. The electrolytic ejector is fluidly connected to the pump to receive fluid from the first reservoir 102 or third reservoir 106, such as water, and electrolytic control valve 1206 fluidly connected to delivery output line 124, such as third delivery output line 124c. Alternatively, delivery output line 124 transfers fluid to electrolyte storage 1205 for use by electrolysis unit 1202.

Electrolysis unit 1202, seen in FIG. 18, receives the water from first reservoir 102 or third reservoir 106 in response to an actuation of electrolysis control valve 1206. The water is provided to the electrolysis unit 1202 from the third reservoir 106 via the third pump 114 or pump 110, whereby valves to other systems are closed. The control valve may be opened or closed by the controller 140 based on the level of the water inside electrolysis unit 1202. Accordingly, a water level sensor is optionally disposed in electrolysis unit 1202. Electrolysis unit 1202 converts water received from the reservoir to hydrogen and oxygen. The generated hydrogen can be used as fuel for powering the engine of vehicle 200.

### Example 19

Fluid delivery system 100 as described in Example 1, 2, 3, or 5 is disposed in vehicle 200, and includes water collection system 1000. Water collection system 1000 includes a plurality of inlet ports 1002 arranged at various locations to facilitate a flow of rainwater falling on the vehicle 200 to a reservoir, for example, first reservoir 102 and/or the third reservoir 106 via a pump or by gravity, as seen in FIG. 19. Alternatively, water from inlet ports 1002 is transported to collection reservoir 1004, for use as non-potable water or the water may be treated for use as potable water. Treatment options include microbial filters, sediment filters, and/or ultraviolet antimicrobial treatment to purify the water. In an embodiment, the ultraviolet light emitting bulbs may be arranged inside first reservoir 102 and third reservoir 106 to prevent growth of bacteria, fungi, microbes, etc., disposed in a line connecting water collection system 1000 to first reservoir 102 and/or third reservoir 106, or disposed in collection reservoir 1004. The treated water may be transferred to third reservoir 106 storing the potable water by a pump or opening a valve (gravity flow).

Water collection system 1000 optionally includes air conditioner capture tubing 1006, allowing collection of the water discharged by the air conditioning system 240 of vehicle 200. Air conditioner capture tubing 1006 runs to capture reservoir 1008, whereby the collected water is transported to collection reservoir 1004, or first reservoir 102 or third reservoir 106 using capture pump 1010. Non-potable water (A) transported to first reservoir 102 may be cleaned using filers and/or UV filter 1115 to clean the water and allow its use as potable water (B). Alcohol (C) is stored in second reservoir 104.

Water collection system 1000 optionally includes a bypass conduit 1111 and a bypass valve 1112 arranged to control the flow of water through the bypass conduit. The bypass valve may be a solenoid actuated valve and when actuated allows the water collected to flow to the ground and thus bypass the first reservoir 102 and the third reservoir 106. A user may actuate the bypass valve (i.e., open the bypass valve) during washing of the vehicle 200 or using the system for hydroponic, and or aeroponic, and or aquaponic use. In an embodiment, the water collection system may collect the water from the atmosphere by cooling the air below its dew point, and thus condensing the moisture present inside the atmospheric air to water.

### Example 20

Although, the mixing valve 136 is contemplated to provide disinfecting liquid at the outlet of first pump 110 to provide the mixture of the water and the disinfecting liquid to various discharge systems. It may be appreciated that the mixture of disinfecting liquid and water may be stored inside the first reservoir 102. In such a case, mixing valve 136 may be omitted. In some embodiments, the at least one hand wash valve 134, the at least one-foot wash valve 154, the at least one harm deterrent valve 304, and the at least one defroster valve 162, each may be a 3 way valve with two inlet ports, one connected with first pump 110 and the other connected with the second pump 112. In such a case, the metering valve 136 may also be omitted.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of a drug delivery system, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. A fluid delivery system, comprising:
at least one reservoir, wherein the at least one reservoir is configured to retain a fluid and has at least an output;
at least one pump in fluid communication with the output of the at least one reservoir and configured to pump the fluid;
at least one ejector in fluid communication with the at least one pump;
at least one valve arranged in fluid communication with the at least one pump and adapted to control a flow of the at least one fluid from the at least one pump to the at least one ejector; and
a controller coupled to the at least one pump and the at least one valve;
wherein the controller is configured to activate the at least one pump and the at least one valve upon activation of the fluid delivery system by a user.

2. The fluid delivery system of claim 1, wherein:
the at least one reservoir further comprises a first reservoir to store a first fluid and a second reservoir to store a second fluid,
wherein the second reservoir is in fluid communication with the least one ejector and in fluid communication with a flow of the first fluid; and
the at least one pump includes a first pump configured to pump the first fluid from the first reservoir.

3. The fluid delivery system of claim 2, further comprising at least a second valve disposed in fluid communication with the second reservoir and coupled to the controller to activate flow of the second fluid into the first fluid upon activation of the fluid delivery system by a user.

4. The fluid delivery system of claim 2, further comprising a second pump in fluid communication with the second reservoir;
wherein the second pump is configured to pump the second fluid from the second reservoir; and
wherein the second pump is coupled to the controller to activate flow of the second fluid into the first fluid upon activation of the fluid delivery system by a user.

5. The fluid delivery system of claim 1, wherein the at least a first valve is a metering valve, mixing valve, or solenoid valve.

6. The fluid delivery system of claim 3, wherein the at least a second valve is a metering valve, a mixing valve, or venturi mixing chamber, and wherein the at least a second valve is disposed in fluid communication with flow of the first fluid.

7. The fluid delivery system of claim 2, wherein the at least one ejector is a hand wash ejector, foot wash ejector, harm deterrent ejector, defrost ejector, or a combination thereof;
wherein the fluid delivery system further comprises:
a mixing valve or mixing chamber disposed between the first reservoir and the second reservoir, and in fluid communication with the first reservoir and the second reservoir;
an output line in fluid communication with the at least one pump and an ejector disposed on the wash line and configured to eject a mixture of the first fluid and the second fluid liquid;
wherein the second fluid is an alcohol or alcohol-based disinfecting fluid;
wherein the hand wash ejector is disposed on an exterior face of a vehicle body panel, an exterior vehicle mirror, a vehicle fueling port, a vehicle charging port, a vehicle bed, or a combination thereof;
wherein the hand wash ejector further comprises a switch, button, or trigger disposed adjacent to the hand wash ejector and in electrical communication with the controller;
a nozzle or port disposed on an end of the output line of the fluid delivery system and adapted to provide a spray of fluid;
wherein the foot wash ejector is disposed on an exterior face of a vehicle door sill, an exterior vehicle wheel well, an exterior bumper, or a combination thereof;
wherein the foot wash ejector further comprises a switch, button, or trigger disposed adjacent to the foot wash ejector and in electrical communication with the controller;
a nozzle or port disposed on an end of the output line of the fluid delivery system and adapted to provide a spray of fluid;
wherein the harm deterrent ejector is disposed on an exterior face of a vehicle body panel;
wherein the harm deterrent ejector further comprises a switch, button, or trigger in radio communication, electromagnetic communication, or electrical communication with the controller;
a nozzle or port disposed on an end of the output line of the fluid delivery system and adapted to provide a spray of fluid; and
wherein the at least one defrost ejector is disposed on an exterior face of a vehicle body panel and configured to eject a mixture of the first fluid and the second fluid liquid;
wherein the defrost ejector further comprises a switch, button, or trigger in radio communication, electromagnetic communication, or electrical communication with the controller;
a nozzle or port disposed on an end of the output line of the fluid delivery system and adapted to provide a spray of fluid.

8. The fluid delivery system of claim 7, further comprising an air dryer, wherein the air dryer comprises:
an air pump disposed in the vehicle and coupled to the controller, wherein the controller activates the air pump upon activation of the fluid delivery system by a user;
a channel in fluid communication with the air pump and configured to transfer air from the air pump to a dryer exhaust;
the dryer exhaust disposed on an exterior panel of the vehicle, a door pillar, or sill of the vehicle, or an interior panel of the vehicle.

9. The fluid delivery system of claim 7, further comprising a brush having a plurality of bristles, wherein the brush is disposed on an exterior panel of the vehicle, a door pillar of the vehicle, or an interior panel of the vehicle.

10. The fluid delivery system of claim 1, wherein the at least one ejector is at least one user wash ejector, fire retardant ejector, at least one vehicle wash system, at least one cloth washing ejector, at least one eye wash ejector, at least one drinking ejector, or at least one ice maker;
wherein the fluid delivery system further comprises:
an output line in fluid communication with the at least one pump and an ejector disposed on the wash line and configured to eject the first fluid;
wherein the user wash ejector is disposed on an exterior body panel of the vehicle or in a compartment disposed on the exterior body panel;
wherein the at least one cloth washing ejector further comprises a switch, button, or trigger disposed adjacent to the at least one cloth washing ejector and in electrical communication with the controller;
a nozzle or port disposed on an end of the output line of the fluid delivery system and adapted to provide a spray of fluid;
wherein the at least one fire retardant ejector is disposed in a motor compartment or interior passenger compartment and aligned to provide a fluid to a predetermined location in the vehicle or around the vehicle;
wherein the at least one cloth washing further comprises a switch or sensor in electrical communication with the controller;
a nozzle or port disposed on an end of the output line of the fluid delivery system and adapted to provide a spray of fluid;
wherein the at least one vehicle wash ejector is a plurality of vehicle wash ejectors disposed on a plurality of exterior panels of the vehicle and aligned to provide a fluid substantially on the exterior of the vehicle;
wherein the at least one vehicle wash ejector further comprises a switch, button, or trigger in electrical communication with the controller;
a nozzle or port disposed on an end of the output line of the fluid delivery system and adapted to provide a spray of fluid;
wherein the at least one cloth washing ejector is disposed on an exterior panel of the vehicle and aligned to provide fluid to a user;
wherein the at least one cloth washing ejector further comprises a switch, button, or trigger disposed adjacent to the at least one cloth washing and in electrical communication with the controller;
a nozzle or port disposed on an end of the output line of the fluid delivery system;
wherein the eye wash ejector is disposed on an exterior body panel of the vehicle, in a compartment disposed on the exterior body panel, or an interior passenger compartment;
wherein the at least one eye wash ejector further comprises a switch, button, or trigger disposed adjacent to the at least one eye wash ejector and in electrical communication with the controller;
a nozzle or port disposed on an end of the output line of the fluid delivery system;
wherein the at least one drinking ejector is disposed on an interior passenger compartment;
wherein the at least one drinking ejector further comprises a switch, button, or trigger disposed adjacent to the at least one drinking ejector and in electrical communication with the controller;
a nozzle or port disposed on an end of the output line of the fluid delivery system; and
wherein the ice maker is disposed on an interior passenger compartment;
wherein the ice maker further comprises a heat exchanger in thermal communication with the output line of the fluid delivery system;
a heat pump in communication with the heat exchanger;
a nozzle or port disposed on an end of the output line of the fluid delivery system.

11. The fluid delivery system of claim 10, wherein the plurality of vehicle wash ejectors are rotatably disposed on the exterior panels of the vehicle, extendably disposed on the exterior panels of the vehicle, or a combination thereof.

12. The fluid delivery system of claim 10, further comprising a chemical fire retardant in fluid communication with the at least one fire retardant ejector.

13. The fluid delivery system of claim 1, further comprising a water capture system, wherein the water capture system comprises:
at least one water collection port disposed on an exterior panel of the vehicle or in the motor compartment of the vehicle;
a capture collection reservoir in fluid communication with the at least one water collection port, wherein the capture collection reservoir is configured to retain a fluid and has at least one input and at least one output.

14. The fluid delivery system of claim 1, further comprising a security system, wherein the security system comprises:
a remote reader sensor disposed in the vehicle having a radio frequency transmitter and radio frequency receiver or radio frequency transceiver;
wherein the remote reader sensor is coupled to the controller;
a user security device, wherein the user security device comprises at least a radio frequency transmitter having a preset code assigned in the user security device, wherein the user security device transmits the preset code upon activation;
wherein the remote reader sensor transmits an activation signal to the controller upon receiving the preset code.

15. The fluid delivery system of claim 1, further comprising a user-activated switch disposed on an interior panel or the vehicle, user-activated switch disposed on a vehicle door pillar, or a user user-activated switch disposed on an exterior panel of the vehicle;
wherein the sensor transmits an activation signal to the controller upon activation of the user-activated switch.

16. The fluid delivery system of claim 1, further comprising a sensor disposed on an exterior panel of the vehicle, wherein the sensor is a motion sensor, an ultrasonic sensor, or a passive infrared sensor;
wherein the sensor transmits an activation signal to the controller upon activation of the sensor.

17. The fluid delivery system of claim 1, wherein the at least one reservoir is configured to store potable water;
wherein the at least one ejector is at least one drinking water ejector disposed in the interior of the vehicle and adapted to eject potable water, and
the at least one valve includes at least one drinking water valve arranged in fluid communication with at least one pump to control a flow of the potable water from the at least one pump to the at least one drinking water ejector.

18. The fluid delivery system of claim 1, wherein
the at least one ejector is at least one cooking fuel ejector in fluid communication with a reservoir and adapted to eject alcohol, and
at least one valve includes at least one cooking valve arranged in fluid communication with the at least one pump to control a flow of alcohol from the at least one pump to the at least one cooking fuel ejector.

19. The fluid delivery system of claim 1, further comprising a manifold in fluid communication with the at least one reservoir, wherein the manifold comprises an input port and a plurality of output ports in fluid communication with the input port.
wherein the manifold is configured to split fluid between the plurality of output ports.
